(19) United States
(12) Patent Application Publication
(10) Pub. No.: US 2024/0104802 A1
(43) Pub. Date: Mar. 28, 2024

(54) MEDICAL IMAGE PROCESSING METHOD AND APPARATUS AND MEDICAL DEVICE

(71) Applicant: GE Precision Healthcare LLC, Waukesha, WI (US)

(72) Inventors: Bingjie Zhao, Beijing (CN); Weiwei Xing, Beijing (CN); Xueli Wang, Beijing (CN)

(21) Appl. No.: 18/475,018

(22) Filed: Sep. 26, 2023

(30) Foreign Application Priority Data
Sep. 27, 2022 (CN) 202211179580.6

(51) Int. Cl. G06T 11/00; A61B 6/03; G16H 30/40

(52) U.S. Cl. CPC G06T 11/006; A61B 6/032; G16H 30/40; G06T 2210/41; G06T 2211/441

(57) ABSTRACT

Embodiments of the present application provide a medical image processing method and apparatus and a medical device, the medical image processing apparatus including an acquisition unit, configured to acquire raw local projection data obtained by a detector after an object to be examined is scanned, a processing unit, configured to recover the raw local projection data to estimate first global data, a determination unit, configured to determine second global data according to the raw local projection data and the first global data, and a reconstruction unit, configured to reconstruct the second global data to obtain a diagnostic image.

### CROSS-REFERENCE TO RELATED APPLICATIONS

This application claims priority to Japanese Application No. 202211179580.6, filed on Sep. 27, 2022, the disclosure of which is incorporated herein by reference in its entirety.

### TECHNICAL FIELD

Embodiments of the present application relate to the technical field of medical devices, and relate in particular to a medical image processing method and apparatus and a medical device.

### BACKGROUND

In the process of computed tomography (CT), a detector is used to acquire data of X-rays passing through an object to be examined, and then the acquired X-ray data is processed to obtain projection data. The projection data may be used to reconstruct a CT image. Complete projection data can be used to reconstruct an accurate CT image for diagnosis.

It should be noted that the above description of the background is only for the convenience of clearly and completely describing the technical solutions of the present application, and for the convenience of understanding of those skilled in the art.

### SUMMARY

Embodiments of the present application provide a medical image processing method and apparatus and a medical device.

According to an aspect of the embodiments of the present application, a medical image processing method is provided. The method includes acquiring raw local projection data obtained by a detector after an object to be examined is scanned, recovering the raw local projection data to estimate first global data, determining second global data according to the raw local projection data and the first global data, and reconstructing the second global data to obtain a diagnostic image.

According to an aspect of the embodiments of the present application, a medical image processing apparatus is provided, including an acquisition unit configured to acquire raw local projection data obtained by a detector after an object to be examined is scanned, a processing unit configured to recover the raw local projection data to estimate first global data, a determination unit configured to determine second global data according to the raw local projection data and the first global data, and a reconstruction unit configured to reconstruct the second global data to obtain a diagnostic image.

According to an aspect of the embodiments of the present application, a medical device is provided, the medical device comprising the medical image processing apparatus according to the preceding aspect.

One of the benefits of the embodiments of the present application is that, the second global data is determined according to the raw local projection data and the first global data obtained by recovering the raw local projection data, and the second global data is reconstructed to obtain the diagnostic image, hence detector data can be recovered, and the impact of artifacts due to data truncation can be reduced, and image quality can be guaranteed when the detector is incomplete.

With reference to the following description and drawings, specific implementations of the embodiments of the present application are disclosed in detail, and the means by which the principles of the embodiments of the present application can be employed are illustrated. It should be understood that the embodiments of the present application are not therefore limited in scope. Within the scope of the spirit and clauses of the appended claims, the embodiments of the present application comprise many changes, modifications, and equivalents.

### BRIEF DESCRIPTION OF THE DRAWINGS

The included drawings are used to provide further understanding of embodiments of the present application, which constitute a part of the description and are used to illustrate embodiments of the present application and explain the principles of the present application together with textual description. Evidently, the drawings in the following description are merely some embodiments of the present application, and a person of ordinary skill in the art may obtain other embodiments according to the drawings without involving inventive skill. In the drawings:

FIG. 1 is example diagrams of incomplete detectors of embodiments of the present application;

FIG. 2 is an example diagram of a complete detector of the embodiments of the present application;

FIG. 3 is an example diagram of a cross-detector of the embodiments of the present application;

FIG. 4 is a schematic diagram of a medical image processing method of the embodiments of the present application;

FIG. 5 is a schematic diagram of an implementation of operation 402 of the embodiments of the present application;

FIG. 6 is a schematic diagram of an implementation of operation 403 of the embodiments of the present application;

FIG. 7 is image fusion schematic diagrams of the embodiments of the present invention;

FIG. 8 is a schematic diagram of a process for acquiring a diagnostic image of the embodiments of the present invention;

FIG. 9 is a schematic diagram of another implementation of operation 402 of the embodiments of the present application;

FIG. 10 is a schematic diagram of the process for acquiring a diagnostic image of the embodiments of the present invention;

FIG. 11 is a schematic diagram of a comparison of diagnostic images obtained in the embodiments of the present invention;

FIG. 12 is a schematic diagram of a neural network model training method of the embodiments of the present application;

FIG. 13A is a schematic diagram of a first training reconstructed image in polar coordinates of the embodiments of the present invention;

FIG. 13B is a schematic diagram of a first training reconstructed image in rectangular coordinates of the embodiments of the present application;

FIG. 13C is a schematic diagram of a first partial training image in rectangular coordinates of the embodiments of the present application;

FIG. 14A is a schematic diagram of a second training reconstructed image in polar coordinates of the embodiments of the present invention;

FIG. 14B is a schematic diagram of a second training reconstructed image in rectangular coordinates of the embodiments of the present application;

FIG. 14C is a schematic diagram of a second partial training image in rectangular coordinates of the embodiments of the present application;

FIG. 15 is a schematic diagram of a method for training a first neural network model of the embodiments of the present application;

FIG. 16A is a schematic diagram of a first training sinogram of the embodiments of the present invention;

FIG. 16B is a schematic diagram of a second training sinogram of the embodiments of the present application;

FIG. 17 is a schematic diagram of a method for training a second neural network model of the embodiments of the present application;

FIG. 18 is a schematic diagram of a medical image processing apparatus of the embodiments of the present application;

FIG. 19 is a schematic diagram of an implementation of a processing unit 1802 of the embodiments of the present application;

FIG. 20 is a schematic diagram of an implementation of a determination unit 1803 of the embodiments of the present invention;

FIG. 21 is a schematic diagram of another implementation of the processing unit 1802 of the embodiments of the present application;

FIG. 22 is a schematic diagram of a configuration of a training unit 1805 of the embodiments of the present application;

FIG. 23 is a schematic diagram of a neural network model training apparatus of the embodiments of the present application;

FIG. 24 is a schematic diagram of a medical image processing device of the embodiments of the present application; and

FIG. 25 is a schematic diagram of a medical device according to the embodiments of the present application.

### DETAILED DESCRIPTION

The foregoing and other features of the embodiments of the present application will become apparent from the following description and with reference to the drawings. In the description and drawings, specific embodiments of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first” and “second” and so on are used to distinguish different elements from one another by their title, but do not represent the spatial arrangement, temporal order, or the like of the elements, and the elements should not be limited by said terms. The term “and/or” includes any one of and all combinations of one or more associated listed terms. The terms “comprise”, “include”, “have”, etc., refer to the presence of stated features, elements, components, or assemblies, but do not exclude the presence or addition of one or more other features, elements, components, or assemblies.

In the embodiments of the present application, the singular forms “a”, “the”, and the like include plural forms, and should be broadly understood as “a type of” or “a class of” rather than limited to the meaning of “one”. In addition, the term “said” should be understood as including both the singular and plural forms, unless otherwise clearly specified in the context. In addition, the term “according to” should be construed as “at least in part according to . . . ”, and the term “based on” should be construed as “at least in part based on . . . ”, unless otherwise clearly specified in the context.

The features described and/or illustrated for one embodiment may be used in one or more other embodiments in an identical or similar manner, combined with features in other embodiments, or replace features in other embodiments. The term “include/comprise” when used herein refers to the presence of features, integrated components, steps, or assemblies, but does not exclude the presence or addition of one or more other features, integrated components, steps, or assemblies.

The device described herein for obtaining medical imaging data may be applicable to various medical imaging modalities, including, but not limited to, computed tomography (CT) devices, or any other suitable medical imaging devices.

The system for obtaining medical images may include the aforementioned medical imaging device, and may include a separate computer device connected to the medical imaging device, and may further include a computer device connected to an Internet cloud, the computer device being connected by means of the Internet to the medical imaging device or a memory for storing medical images. The imaging method may be independently or jointly implemented by the aforementioned medical imaging device, the computer device connected to the medical imaging device, and the computer device connected to the Internet cloud.

For example, the CT scan uses X-rays to carry out continuous profile scans around a certain part of a scanned object, and detectors receive the X-rays that pass through said plane and transform the same into visible light or directly convert a received photon signal and then reconstruct an image by means of a series of processes. MRI is based on the principle of nuclear magnetic resonance of atomic nuclei, and forms an image by means of reconstruction by transmitting radio frequency pulses to the scanned object and receives electromagnetic signals emitted from the scanned object.

In addition, a medical imaging workstation may be disposed locally at the medical imaging device. That is, the medical imaging workstation is disposed near to the medical imaging device, and the medical imaging workstation and medical imaging device may be located together in a scanning room, an imaging department, or in the same hospital. A medical image cloud platform analysis system may be located away from the medical imaging device, for example, arranged at a cloud end that is in communication with the medical imaging device.

As an example, after a medical institution completes an imaging scan by using the medical imaging device, scan data is stored in a storage device. The medical imaging workstation may directly read the scan data and perform image processing by means of a processor thereof. As another example, the medical image cloud platform analysis system may read a medical image in the storage device by means of remote communication to provide “software as a service (SaaS).” SaaS can exist between hospitals, between a hospital and an imaging center, or between a hospital and a third-party online diagnosis and treatment service provider.

In the embodiments of the present application, the term “object to be examined” may include any object being imaged. In some embodiments, the term “projection data” is interchangeable with “projection image” and “sinogram”.

The detector is an extremely important and high-priced component in CT, and the quality of the detector may affect the quality of the final imaging. The CT detector typically includes a plurality of detector modules. The detector functions to convert an incident invisible X-light into a scintillating crystal or fluorescent substance of visible light, so as to complete subsequent imaging. Each detector module has a photoelectric sensor assembly, which records X-rays that are incident on the CT detector modules, and converts them into an electrical signal, so as to facilitate subsequent processing of the electrical signal.

In the prior art, a plurality of detector modules are arranged in an array in a CT casing. The inventor found that in an actual application scenario, sometimes the final imaging could be achieved without use of a complete detector. For example, in a cardiac scan, only an image of a center of 25-30 cm would be sufficient to cover the cardiac region. Therefore, in order to reduce costs, an incomplete detector with partial off-center detector modules removed from a plurality of detector modules arranged in an array (a complete detector) may be used for scanning. The positions of the removed partial off-center detector modules may be symmetric or asymmetric, and the embodiments of the present application are not limited thereto.

An exemplary illustration of a complete detector and incomplete detector is first given below.

The projection image or data obtained by a complete detector is complete or, in other words, global, and the projection data or image obtained by the incomplete detector is incomplete or, in other words, local. In addition, for convenience of illustration, in the embodiments of the present application, the projection data or image that should have otherwise been obtained by the removed detector modules is referred to as missing data or a missing image. An incomplete detector is used for scanning and may obtain raw local projection data, but the missing data is also used for filtering and backward projection of the raw local projection data of adjacent positions thereto in the image reconstruction process, and therefore the incorrectness of the missing data may cause CT values within a scanning field to drift, and cause truncation artifacts to appear in an image, resulting in a distorted and inaccurate reconstructed image.

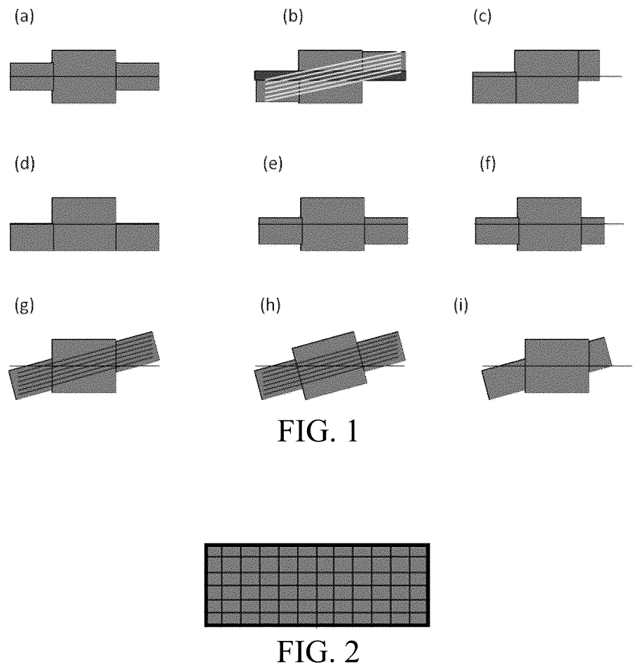

FIG. 1 is example diagrams of incomplete detectors of the embodiments of the present application, and FIG. 2 is an example diagram of a complete detector of the embodiments of the present application. As shown in FIG. 2, global projection data of a complete rectangular region may be obtained by the complete detector. As shown in FIG. 1(a), partial detector modules in four corners of a plurality of detector modules arranged in an array may be symmetrically removed, for example, half of the detector modules may be removed and half of the detector modules may be left, and the incomplete detector in FIG. 1(a) may be referred to as a cross-detector. As shown in FIG. 1(b), FIG. 1(c), FIG. 1(d), FIG. 1(e), FIG. 1(f), FIG. 1(g), FIG. 1(h), and FIG. 1(i), any partial detector modules in at least one among four corners of a plurality of detector modules arranged in an array may be asymmetrically removed, and local projection data of a central region may be retained in projection data of the detector. The embodiments of the present invention are not limited thereto. The incomplete detector may also be a fence-shaped detector or others, and examples will not be listed herein one by one.

In some embodiments, the size of the central region may be determined according to a region of interest, that is, when the detector modules are removed, it must be guaranteed that the remaining detectors in the central region are able to acquire projection data of the region of interest. As for which off-center detector modules are removed, this can be determined as needed.

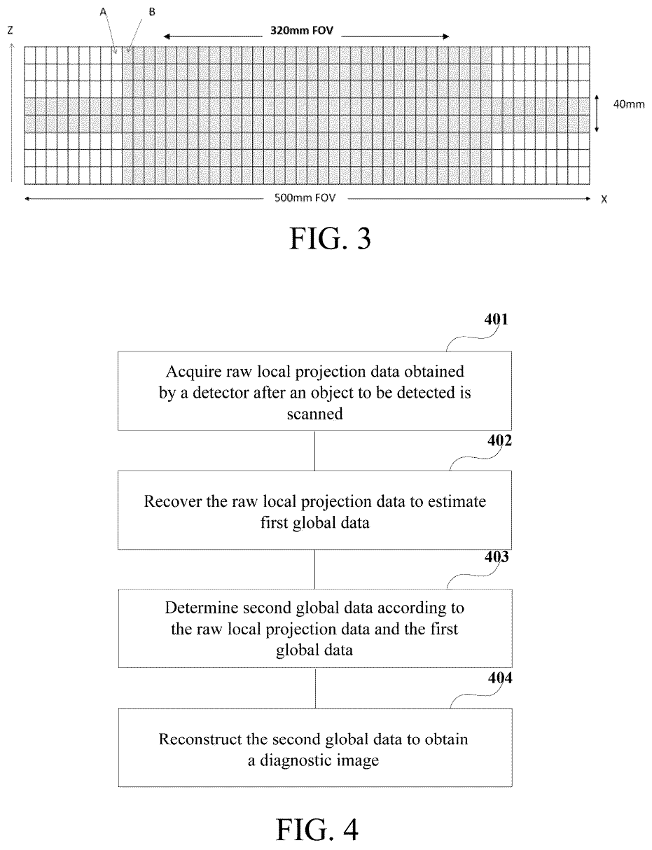

In the following embodiments, for convenience, an illustration is given by taking a cross-detector as an example. FIG. 3 is a schematic diagram of a cross-detector of the embodiments of the present application. As shown in FIG. 3, when the dimensions of the complete detector is 500 mm×160 mm, the cross-detector retains detector modules in a central region of 320 mm in an X direction and detector modules in a central region of 40 mm in a Z direction. This is merely an example illustration herein, and the embodiments of the present application are not limited thereto.

The inventor further found that, if the incomplete detector is used for scanning, incomplete image data may reduce the image quality, and if a blank (missing) data portion is simply filled with 0 or by other traditional methods, CT values within a scanning field may be caused to drift and truncation artifacts may occur in an image, resulting in a distorted and inaccurate reconstructed image.

In view of at least one among the above technical problems, a medical image processing method and apparatus and medical device are provided in the embodiments of the present application, in which second global data is determined according to raw local projection data as well as first global data obtained by recovering the raw local projection data, and the second global data is reconstructed to obtain a diagnostic image, hence detector data can be recovered, and the impact of the artifacts due to data truncation can be reduced, and image quality can be guaranteed when the detector is incomplete.

The following is a specific description of an embodiment of the present invention with reference to the accompanying drawings.

Embodiments of the present application provide a medical image processing method. FIG. 4 is a schematic diagram of a medical image processing method of the embodiments of the present application. As shown in FIG. 4, the method includes acquiring raw local projection data obtained by a detector after an object to be examined is scanned (block 401), recovering the raw local projection data to estimate first global data (block 402), determining second global data according to the raw local projection data and the first global data (block 403), and reconstructing the second global data to obtain a diagnostic image (block 404).

In some embodiments, scan data may be acquired by means of various medical imaging modalities, including, but not limited to, data obtained by computed tomography (CT) or other suitable medical imaging techniques. The data may be two-dimensional data or three-dimensional data or four-dimensional data, and the embodiments of the present application are not limited thereto.

In some embodiments, the detector is an incomplete detector with partial off-center detector modules removed from a plurality of detector modules arranged in an array (a complete detector), for example the incomplete detector(s) in FIG. 1 or FIG. 3. The remaining detector modules in the center position are used to scan the object to be examined, the scanning including scanning of a region of interest. The region of interest may be set as needed, for example, the region of interest is the cardiac region.

In some embodiments, in 401, the object to be examined is scanned, data passing through the object to be examined is acquired by using the incomplete detector, and then the acquired data is processed to obtain the raw local projection data. Please refer to related technology for details, which will not be described herein again.

In some embodiments, in 402, the raw local projection data may be recovered to obtain estimated missing data, and the first global data is determined according to the estimated missing data and the raw local projection data. For example, the raw local projection data may be recovered by using a deep learning method to estimate the first global data. That is, the raw local projection data is processed to obtain a first reconstructed image or a first sinogram, and the first reconstructed image or the first sinogram is inputted into a pre-trained neural network model, so as to estimate the first global data. The missing data or image of the incomplete detector is recovered in an image domain or a sinusoidal domain by using the deep learning method, and the first global data includes a first global image in the image domain or a first global sinogram in the sinusoidal domain.

In some embodiments, in 403, the raw local projection data and the first global data may be fused to obtain the second global data. When the first global data is the first global image in the image domain, in 403, it is required to perform a forward projection on the first global data and then fuse the resulting data with the raw local projection data to obtain the second global data. In 404, the second global data is reconstructed to obtain the diagnostic image.

The following illustrates operations 402 to 404 by taking the image domain and the sinusoidal domain as examples, respectively.

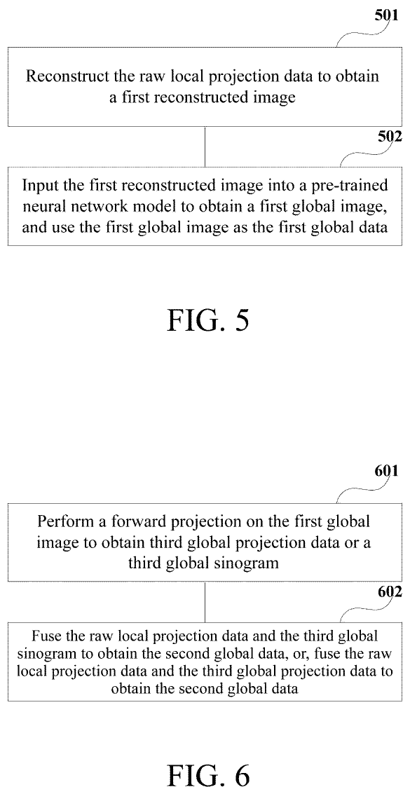

In some embodiments, the missing data or image of the incomplete detector may be recovered in the image domain by using the deep learning method. FIG. 5 is a schematic diagram of an implementation of operation 402 of the embodiments of the present application. As shown in FIG. 5, operation 402 includes reconstructing the raw local projection data to obtain a first reconstructed image (block 501), and inputting the first reconstructed image into a pre-trained neural network model to obtain a first global image, and using the first global image as the first global data (block 502).

In some embodiments, in 501, the raw local projection data may be processed to obtain the first sinogram, and the first sinogram is image-reconstructed to obtain a first reconstructed image in the image domain, or the raw local projection data may also be used directly to perform image reconstruction to obtain the first reconstructed image in the image domain. During the image reconstruction, first filling data may be filled in the position of the missing image or data, and the first filling data is subjected to an image reconstruction algorithm in conjunction with the raw local projection data to obtain the first reconstructed image in the image domain, the first reconstructed image having truncation artifacts therein. The image reconstruction algorithm may include, for example, a backward projection (FBP) reconstruction method, an adaptive statistical iterative reconstruction (ASIR) method, a conjugate gradient (CG) method, a maximum likelihood expectation maximization (MLEM) method, a model-based iterative reconstruction (MBIR) method, etc. Please refer to related technology for details, and the embodiments of the present application are not limited thereto.

In some embodiments, the first filling data is determined according to projection data acquired by an edge detector module in the detector. For example, the value of the first filling data may be determined according to the raw local projection data of the position of the non-missing data (hereinafter referred to as a second position) that is adjacent to the position of the missing data filled with the first filling data (hereinafter referred to as a first position). First filling data filled in different first positions are the same or different. For example, the first filling data of the first position may be equal to the raw local projection data of one second position, or equal to an average or maximum or minimum value of the raw local projection data of a plurality of second positions. As shown in FIG. 3, the first filling data filled in a first position A may be equal to the raw local projection data of a second position B. Alternatively, the first data may be a fixed value. For example, the fixed value may be 0, and the embodiments of the present application are not limited thereto.

In some embodiments, in 502, the missing data or image of the incomplete detector may be recovered by using the pre-trained neural network model to remove the artifacts in the image caused by the incomplete detector. For the neural network model (in the image domain, also referred to as a first neural network model), the input is the first reconstructed image obtained in 501, and the output is the first global image, or the output is a difference image between the first global image and the first reconstructed image. When the output is the difference image, it is required to merge the difference image and the first reconstructed image to obtain the first global image. As for how the neural network model is pre-trained, it will be described in the following embodiments.

In some embodiments, in 403, the second global data is determined in the image domain according to the raw local projection data and the first global data. FIG. 6 is a schematic diagram of an implementation of operation 403 of the embodiments of the present application. As shown in FIG. 6, operation 403 includes performing a forward projection on the first global image to obtain third global projection data or a third global sinogram (block 601), and fusing the raw local projection data and the third global sinogram to obtain the second global data, or fusing the raw local projection data and the third global projection data to obtain the second global data (block 602).

In some embodiments, since the detector is incomplete, the missing projection data or missing sinogram cannot be directly obtained by scanning. In 601, a forward projection (or frontward projection) is performed on the first global image to obtain third global projection data in a projection domain or a third global sinogram in the sinusoidal domain, the third global projection data or the third global sinogram comprising projection data or a sinogram corresponding to the estimated missing image recovered using the deep learning network.

In some embodiments, due to in the third global projection data or the third global sinogram, there is a problem of unsmoothness and discontinuity between the missing projection data and the raw local projection data (or between the missing sinogram and a local sinogram corresponding to the raw local projection data). For this problem, in 602, the third global projection data or the third global sinogram is amended by using the raw local projection data obtained by scanning, i.e., a sinogram corresponding to the raw local projection image (a first sinogram) and the third global sinogram are fused to obtain the second global sinogram, and the second global sinogram is used as the second global data; or the raw local projection data and the third global projection data are fused to obtain the second global projection data, and the second global projection data is used as the second global data.

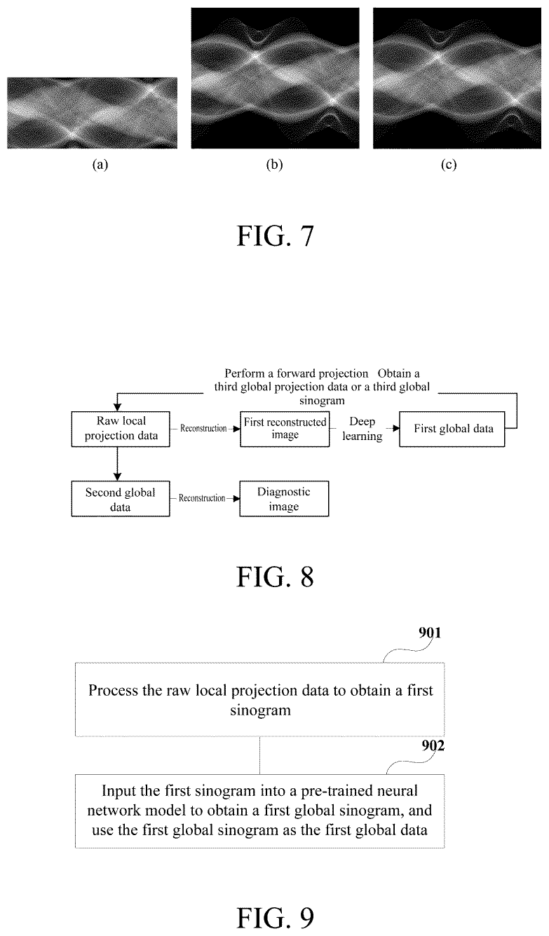

FIG. 7 is image fusion schematic diagrams of the embodiments of the present invention, wherein FIG. 7(a) is a first sinogram obtained by an incomplete detector, FIG. 7(b) is a third global sinogram, and FIG. 7(c) is a result of fusing FIG. 7(a) and FIG. 7(b). It can be seen that the second global data is more smooth than the first global data, and steps in the first global data can be removed. The image fusion processing includes calculating the difference of an overlapping portion between the first sinogram (the raw local projection data) and the third global sinogram (the third global projection data), compensating (adding) the difference to the third global sinogram (the third global projection data), and then replacing the first sinogram (the raw local projection data) into the third global sinogram (the third global projection data) in a corresponding position. Therefore, the missing data can be amended by calculating the difference between estimated data (global) and actual scan data (local) in conjunction with sinusoidal domain and image domain information, so as to further ensure image quality.

In some embodiments, in operation 404, the second global data (the second global sinogram or the second global projection data) is reconstructed to obtain the diagnostic image. Upon reconstruction, only an image within a field of view (FOV) corresponding to an incomplete detector, for example the incomplete detector shown in FIG. 3, is reconstructed, and the diagnostic image is only an image within the range of 320 mm of a display field (DFOV). In contrast, the aforementioned first reconstructed image, and the first and second training reconstructed images in the following embodiments, are all reconstructed images within a field of view (FOV) corresponding to a complete detector, for example, the complete detector shown in FIG. 2, and the aforementioned first reconstructed image, and the first and second training reconstructed images in the following embodiments, are all images in the range of 500 mm of a display field (DFOV). The image reconstruction algorithm may include, for example, a back projection (FBP) reconstruction method, an adaptive statistical iterative reconstruction (ASIR) method, a conjugate gradient (CG) method, a maximum likelihood expectation maximization (MLEM) method, a model-based iterative reconstruction (MBIR) method, etc. Please refer to related technology for details, and the embodiments of the present application are not limited thereto.

FIG. 8 is a schematic diagram of a process for acquiring a diagnostic image of the embodiments of the present application. As shown in FIG. 8, operation 401 is first performed to obtain raw local projection data; operation 402 is performed to reconstruct the raw local projection data to obtain a first reconstructed image, and to input the first reconstructed image into a deep learning neural network model to estimate first global data (a first global image); operation 403 is performed to perform a forward projection on the first global data (to obtain a third global projection data or a third global sinogram) and then fuse the resulting data with the raw local projection data to obtain a second global data; and operation 404 is performed to reconstruct the second global data to obtain a diagnostic image.

In some embodiments, the missing data or image of the incomplete detector may be recovered in the sinusoidal domain by using the deep learning method. FIG. 9 is a schematic diagram of an implementation of operation 402 of the embodiments of the present application. As shown in FIG. 9, operation 402 includes processing the local projection data to obtain a first sinogram (block 901), and inputting the first sinogram into a pre-trained neural network model to obtain a first global sinogram, and using the first global sinogram as the first global data (block 902).

In some embodiments, in 901, the raw local projection data may be subjected to negative logging (−log) and correction processing to obtain the first sinogram. Optionally, second filling data may be filled in the position of the missing data, and the second filling data is subjected to negative logging (−log) and correction processing in conjunction with the local projection data to obtain the first sinogram, or the first sinogram is generated using a three-dimensional interpolation algorithm. Please refer to related technology for details. The difference between operation 501 is that it is not required to reconstruct the first sinogram or the raw local projection data in the image domain. In 902, the missing data or image of the incomplete detector may be recovered by using the neural network model (in the sinusoidal domain, also referred to as a second neural network model) to remove the artifacts in the image caused by the incomplete detector. For the neural network model, the input is the first sinogram obtained in 901, and the output is the first global image, or the output is a difference image between the first global sinogram and the first sinogram. When the output is the difference image, it is required to merge the difference image and the first sinogram to obtain the first global sinogram. As for how the neural network model is pre-trained, this will be described in the following embodiments. The means for determining the second filling data are similar to the means for determining the first filling data, which will not be described herein again.

In some embodiments, in 403, the second global data is determined in the sinusoidal domain according to the raw local projection data and the first global data. The difference between FIG. 6 is that, due to being in the sinusoidal domain, forward projection is not required to be performed, and the local sinogram and the first global sinogram are directly fused to obtain the second global data.

In some embodiments, since there may be a problem of unsmoothness and discontinuity between the missing sinogram and the local sinogram corresponding to the raw local projection data, in 403, the first global sinogram is amended by using the raw local projection data obtained by scanning, that is, the first sinogram and the first global sinogram are fused to obtain the second global sinogram, and the second global sinogram is used as the second global data. The second global data is smoother than the first global data, and steps in the first global data can be removed. The image fusion includes calculating the difference of an overlapping portion between the first sinogram and the first global sinogram, compensating (adding) the difference to the first global sinogram, and then replacing the first sinogram into the first global sinogram in a corresponding position. Therefore, the missing data can be amended by calculating the difference between estimated data (global) and actual scan data (local) in conjunction with sinusoidal domain and image domain information, to further ensure image quality.

It should be noted that 901 is optional. In 402, it is also possible to directly input the raw local projection data into the pre-trained neural network model to obtain the first global sinogram or the first global projection data, and to use the first global sinogram or the first global projection data as the first global data. In 403, it is also possible to directly fuse the local projection data and the first global projection data to obtain the second global projection data as the second global data. The embodiments of the present application are not limited thereto.

In some embodiments, in 404, the second global data (the second global sinogram or the second global projection data) is reconstructed to obtain the diagnostic image, and upon reconstruction, only the image within the field of view (FOV) corresponding to the incomplete detector, for example the incomplete detector shown in FIG. 3, is reconstructed, and the diagnostic image is only the image in the range of 32 cm of the display field (DFOV). In contrast, the aforementioned first reconstructed image, and the first and second training reconstructed images in the following embodiments, are all reconstructed images within the field of view (FOV) corresponding to the complete detector, for example, the complete detector shown in FIG. 2, and the aforementioned first reconstructed image, and the first and second training reconstructed images in the following embodiments, are all images in the range of 50 cm of the display field (DFOV). The image reconstruction algorithm may include, for example, a back projection (FBP) reconstruction method, an adaptive statistical iterative reconstruction (ASIR) method, a conjugate gradient (CG) method, a maximum likelihood expectation maximization (MLEM) method, a model-based iterative reconstruction (MBIR) method, etc. Please refer to related technology for details, and the embodiments of the present application are not limited thereto.

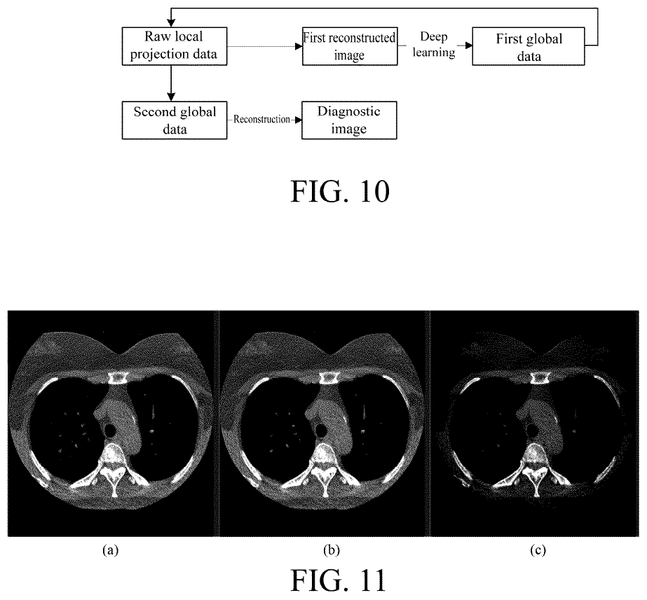

FIG. 10 is a schematic diagram of the process for acquiring a diagnostic image of the embodiments of the present invention. As shown in FIG. 10, operation 401 is first performed to obtain raw local projection data; operation 402 is performed to subject the raw local projection data to negative logging and correction processing to obtain a first sinogram, and to input the first sinogram into a pre-trained neural network model to estimate first global data; operation 403 is performed to fuse the first global data and the raw local projection data to obtain a second global data; and operation 404 is performed to reconstruct the second global data to obtain a diagnostic image.

FIG. 11 is schematic diagrams for a comparison of diagnostic images of the embodiments of the present application, wherein FIG. 11(a) is a schematic diagram of a diagnostic image obtained by means of operations 401-404, FIG. 11(b) is a schematic diagram of a diagnostic image (a metal marker image) obtained using a complete detector, and FIG. 11(c) is a schematic diagram of a diagnostic image that is obtained by using an incomplete detector that is recovered under an existing method. By comparison, the diagnostic image obtained in the embodiments of the present application is closest to the metal marker image. Information reconstructed in the diagnostic image is real information acquired by the incomplete detector and can be used for clinical diagnosis, but the real local projection data needs to be filtered and backward projected in the reconstruction process by using the missing data. By means of the above method of the embodiments of the present application, the detector data can be recovered, and the impact of the artifacts due to data truncation can be reduced, and a higher image quality can be maintained with fewer detectors, reducing product costs.

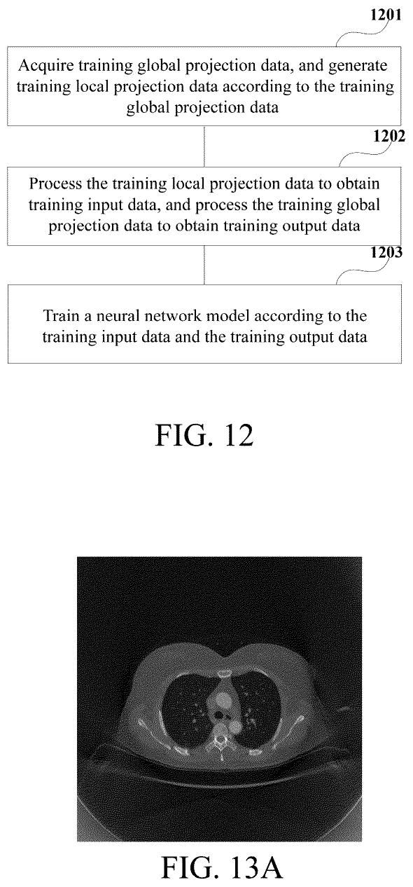

Further provided in the embodiments of the present application is a neural network model training method, which may be two-dimensional or three-dimensional. FIG. 12 is a schematic diagram of a neural network model training method of the embodiments of the present application. As shown in FIG. 12, the method includes acquiring training global projection data, and generating training local projection data according to the training global projection data (block 1201), processing the training local projection data to obtain training input data, and processing the training global projection data to obtain training output data (block 1202), and training a neural network model according to the training input data and the training output data (block 1203).

In some embodiments, the raw local projection data may be recovered by using a pre-trained neural network model to estimate first global data. For example, the missing data or image of an incomplete detector is recovered in an image domain or a sinusoidal domain. The neural network model may be applicable to the image domain (hereinafter referred to as a first neural network model) or the sinusoidal domain (hereinafter referred to as a second neural network model). Explanations are provided below, respectively.

In some embodiments, in 1201, different objects to be examined are scanned, the training global projection data is acquired by using a complete detector corresponding to the incomplete detector in the aforementioned embodiments, and data (missing data) corresponding to removed partial off-center detector modules is deleted, so as to simulate the training local projection data obtained by the incomplete detector.

In some embodiments, in 1202, the training local projection data is reconstructed to obtain a first training reconstructed image as the training input data, and the training global projection data is reconstructed to obtain a second training reconstructed image as the training output data. For example, after filling first filling data therein, the training local projection data is reconstructed to obtain the first training reconstructed image. The first filling data is determined according to projection data acquired by an edge detector module in the detector, or may be a fixed value. That is, the first filling data may be filled in the position of the missing data. The first filling data is image-reconstructed in conjunction with the training local projection data to obtain the first training reconstructed image. That is, the first filling data fills the missing data corresponding to the removed detector modules. As for the reconstruction method, reference may be made to the aforementioned embodiments. As for how to determine the first filling data, please refer to the aforementioned embodiments, which will not be described herein again.

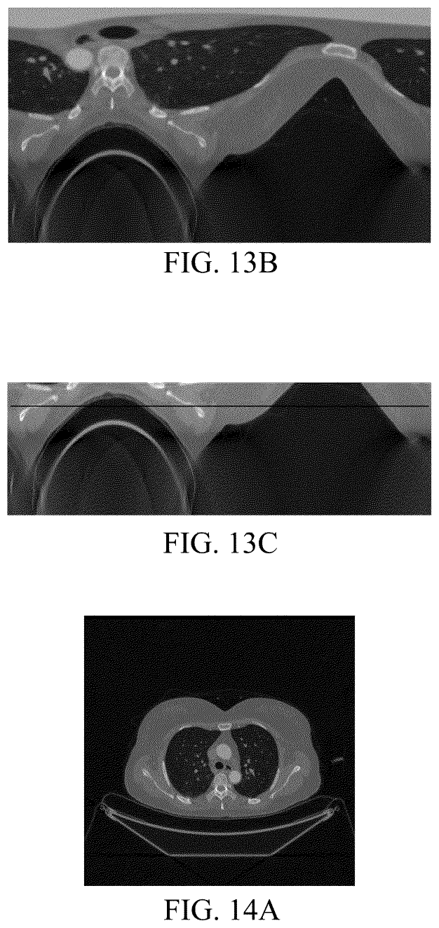

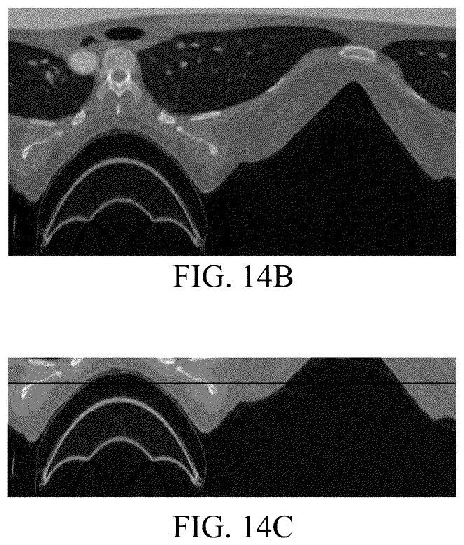

In some embodiments, the first training reconstructed image and the second training reconstructed image may be reconstructed images in polar coordinates. FIG. 13A is a schematic diagram of a first training reconstructed image in polar coordinates of the embodiments of the present application, and FIG. 14A is a schematic diagram of a second training reconstructed image in polar coordinates of the embodiments of the present application, Alternatively, the first training reconstructed image and the second training reconstructed image may also be reconstructed images in a rectangular coordinate system after passing through a coordinate transformation. FIG. 13B is a schematic diagram of a first training reconstructed image in rectangular coordinates of the embodiments of the present application, and FIG. 14B is a schematic diagram of a second training reconstructed image in rectangular coordinates of the embodiments of the present application. The process of the above coordinate transformation can facilitate the centralized extraction of image features to be trained.

And, in 1203, the neural network model is trained according to the first training reconstructed image and the second training reconstructed image. The neural network model is trained by using the training input data as an input to the neural network model, and the training output data as an output from the neural network model, or the neural network model is trained by using the training input data as an input to the neural network model, and the difference between the training output data and the training input data as an output from the neural network model. That is, the neural network model is trained by using the first training reconstructed image as the input to the neural network model, and the second training reconstructed image as the output from the neural network model, or the first neural network model is trained by using the first training reconstructed image as the input to the first neural network model, and a difference image between the second training reconstructed image and the first training reconstructed image as the output from the first neural network model.

In some embodiments, in order to improve the training speed of the first neural network model, reduce the computing amount and improve the image quality, in 1202, it is also possible to take a first partial training image from the first training reconstructed image as the training input data, and take a second partial training image corresponding to the first partial training image from the second training reconstructed image as the training output data.

In some embodiments, the first partial training image and the second partial training image are taken from a first training reconstructed image and a second training reconstructed image in the rectangular coordinate system. The size of the first partial training image is determined according to the position of the removed partial off-center detector modules. For example, in FIG. 3, the position of the removed detector modules is a region of 32 cm-50 cm in the X direction, and the size of the first partial training image is equal to the size of a first image of a region of 320 mm-500 mm in the X direction, or slightly greater than the size of the first image, for example, equal to the size of an image of a region of 300 mm-500 mm in the X direction. The size of the second partial training image is the same as that of the first partial training image. FIG. 13C is a schematic diagram of a first partial training image in rectangular coordinates of the embodiments of the present application, and FIG. 14C is a schematic diagram of a second partial training image in rectangular coordinates of the embodiments of the present application.

And, in 1203, the neural network model is trained according to the first partial training image and the second partial training image. The first neural network model is trained by using the first partial training image as the input to the first neural network model and the second partial training image as the output from the first neural network model, or the first neural network model is trained by using the first partial training image as the input to the first neural network model and a difference image between the second partial training image and the first partial training image as the output from the first neural network model.

In some embodiments, because it is low-frequency information of the missing data that causes the CT values in the scanning field to drift and causes the truncation artifacts to occur in the image, in order to further improve image quality, in 1202, it is also possible to remove high-frequency information in the first partial training image and the second partial training image, and use the first partial training image that has had the high-frequency information removed as the training input data, and the second partial training image that has had the high-frequency information removed as the training output data. The high-frequency information in the first partial training image and the second partial training image may be removed by means of a low-pass filter or a multi-image averaging method. Please refer to the prior art for details, and the embodiments of the present application are not limited thereto.

And, in 1203, the neural network model is trained according to the first and second partial training images that have had the high-frequency information removed. The first neural network model is trained by using the first partial training image that has had the high-frequency information removed as the input to the first neural network model and the second partial training image that has had the high-frequency information removed as the output from the first neural network model, or the first neural network model is trained by using the first partial training image that has had the high-frequency information removed as the input to the first neural network model and a difference image between the second partial training image and the first partial training image that have had the high-frequency information removed as the output from the first neural network model.

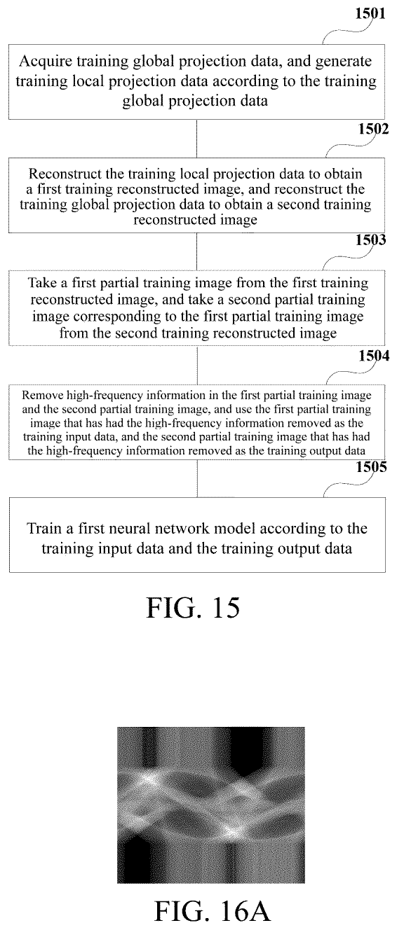

FIG. 15 is a schematic diagram of a method for training a first neural network model of the embodiments of the present application. As shown in FIG. 15, the method includes acquiring training global projection data, and generating training local projection data according to the training global projection data (block 1501), reconstructing the training local projection data to obtain a first training reconstructed image, and reconstructing the training global projection data to obtain a second training reconstructed image (block 1502), taking a first partial training image from the first training reconstructed image, and taking a second partial training image corresponding to the first partial training image from the second training reconstructed image (block 1503), removing high-frequency information in the first partial training image and the second partial training image, and using the first partial training image that has had the high-frequency information removed as the training input data, and using the second partial training image that has had the high-frequency information removed as the training output data (block 1504), and training a first neural network model according to the training input data and the training output data (block 1505).

In the above method, 1503 and 1504 are optional steps. It is possible to directly use the first training reconstructed image in 1502 as the training input data, and the second training reconstructed image as the training output data, or use the first partial training image in 1503 as the training input data, and the second partial training image as the training output data. The embodiments of the present application are not limited thereto.

In some embodiments, in 1201, different objects to be examined are scanned, the training global projection data is acquired by using a complete detector corresponding to the incomplete detector in the aforementioned embodiments, and data (missing data) corresponding to removed partial off-center detector modules is deleted, so as to simulate the training local projection data obtained by the incomplete detector.

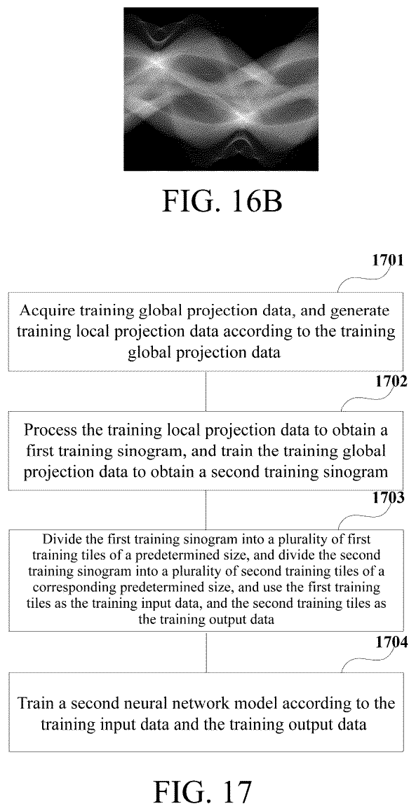

In some embodiments, in 1202, the training local projection data is processed to obtain a first training sinogram as the training input data, and the training global projection data is processed to obtain a second training sinogram as the training output data. For example, in 1202, the training local projection data is processed (subjected to negative logging and correction processing) to obtain the first training sinogram, and the training global projection data is processed (subjected to negative logging and correction processing) to obtain the second training sinogram. Optionally, the training local projection data is processed (subjected to negative logging and correction processing) after second filling data is filled in the training local projection data, to obtain the first training sinogram. That is, the second filling data may be filled in the position of the missing data, and the second filling data is processed in conjunction with the training local projection data to obtain the first training sinogram. Alternatively, the first training sinogram may be generated by using a three-dimensional interpolation method. Please refer to related technology for details. For the means for determining the second filling data, please refer to the method for determining the first filling data, which will not be described herein again. FIG. 16A is a schematic diagram of a first training sinogram of the embodiments of the present application, and FIG. 16B is a schematic diagram of a second training sinogram of the embodiments of the present application.

And, in 1203, the neural network model is trained according to the first training sinogram and the second training sinogram. The neural network model is trained by using the training input data as an input to the neural network model and the training output data as an output from the neural network model, or the neural network model is trained by using the training input data as an input to the neural network model and the difference between the training output data and the training input data as an output from the neural network model. That is, the second neural network model is trained by using the first training sinogram as the input to the second neural network model and the second training sinogram as the output from the second neural network model, or the second neural network model is trained by using the first training sinogram as the input to the second neural network model and the difference between the second training sinogram and the first training sinogram as the output from the second neural network model.

In some embodiments, since there is a large amount of data in a projection domain or the sinusoidal domain, in order to improve the computing speed, in 1202, the first training sinogram may be divided into a plurality of first training tiles of a predetermined size, and the second training sinogram may be divided into a plurality of second training tiles of a corresponding predetermined size, and the first training tiles may be used as the training input data, and the second training tiles may be used as the training output data.

And, in 1203, the neural network model is trained according to the first training tiles and the second training tiles. For example, the second neural network model is trained by using the first training tiles as the input to the second neural network model and the second training tiles as the output from the second neural network model, or the second neural network model is trained by using the first training tiles as the input to the second neural network model and difference images between the second training tiles and the first training tiles as the output from the second neural network model. That is, a pair of training data is dimensionalized as tiles, rather than as a sinogram. The predetermined size may be determined as needed, and the embodiments of the present application are not limited thereto.

FIG. 17 is a schematic diagram of a method for training a second neural network model of the embodiments of the present application. As shown in FIG. 17, the method includes acquiring training global projection data, and generating training local projection data according to the training global projection data (block 1701), processing the training local projection data to obtain a first training sinogram, and processing the training global projection data to obtain a second training sinogram (block 1702), dividing the first training sinogram into a plurality of first training tiles of a predetermined size, and dividing the second training sinogram into a plurality of second training tiles of a corresponding predetermined size, and using the first training tiles as the training input data, and the second training tiles as the training output data (block 1704), and training a second neural network model according to the training input data and the training output data (block 1705).

In the above method, 1703 is an optional step. It is possible to directly use the first training sinogram in 1702 as the training input data, and the second training sinogram as the training output data. The embodiments of the present application are not limited thereto.

In some embodiments, the above first neural network model and second neural network model are composed of an input layer, an output layer, and one or more hidden layers (a convolutional layer, a pooling layer, a normalization layer, etc.) between the input layer and the output layer. Each layer can consist of multiple processing nodes that can be referred to as neurons. For example, the input layer may have neurons for each pixel or set of pixels from a scan plane of an anatomical structure. The output layer may have neurons corresponding to a plurality of predefined structures or predefined types of structures (or organizations therein). Each neuron in each layer may perform processing functions and pass processed medical image information to one neuron among a plurality of neurons in the downstream layer for further processing. That is, “simple” features may be extracted from input data for an earlier or higher-level layer, and then these simple features are combined into a layer exhibiting features of higher complexity. In practice, each layer (or more specifically, each “neuron” in each layer) may process input data as output data for representation by using one or a plurality of linear and/or non-linear transformations (so-called activation functions). The number of the plurality of “neurons” may be constant among the plurality of layers or may vary from layer to layer. For example, neurons in the first layer may learn to recognize structural edges in medical image data. Neurons in the second layer may learn to recognize shapes etc., based on the detected edges from the first layer. The structure of the first neural network model and the second neural network model may be, for example the structure of a VGG16 model, a Unet model, or a Res-Unet model, etc. The embodiments of the present application are not limited thereto, and for the structure of the above models, related technology can be referred to, which will not be described herein again one by one.

The training data (or training image or sinogram) used for neural network model training described above is medical data or a medical image. The pre-trained neural network model may be used to recover missing data that should have otherwise been acquired by the removed detector modules, and the impact of the artifacts due to data truncation may be reduced.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined. For example, the above medical image processing method and the neural network model training method may be implemented separately or in combination, and the embodiments of the present application are not limited thereto.

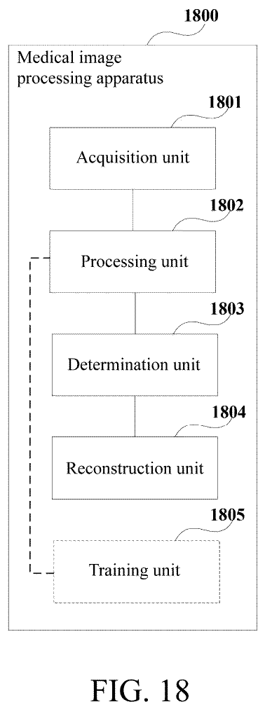

Further provided in the embodiments of the present application is a medical image processing apparatus. FIG. 18 is a schematic diagram of a medical image processing apparatus of the embodiments of the present invention. As shown in FIG. 18, the apparatus 1800 includes an acquisition unit 1801, configured to acquire raw local projection data obtained by a detector after an object to be examined is scanned, a processing unit 1802, configured to recover the raw local projection data to estimate first global data, a determination unit 1803, configured to determine second global data according to the raw local projection data and the first global data, and a reconstruction unit 1804, configured to reconstruct the second global data to obtain a diagnostic image.

In some embodiments, implementations of the acquisition unit 1801, the processing unit 1802, the determination unit 1803, and the reconstruction unit 1804 may refer to 401-404 of the aforementioned embodiments, which will not be described herein again. In some embodiments, the detector is an incomplete detector with partial off-center detector modules removed from a plurality of detector modules arranged in an array. In some embodiments, the processing unit 1802 recovers the raw local projection data to obtain estimated missing data, and determines the first global data according to the estimated missing data and the raw local projection data. In some embodiments, the processing unit 1802 processes the raw local projection data to obtain a first reconstructed image or a first sinogram, and inputs the first reconstructed image or the first sinogram into a pre-trained neural network model to estimate the first global data. In some embodiments, the determination unit 1803 fuses the raw local projection data and the first global data to obtain the second global data. In some embodiments, the determination unit 1803 performs a forward projection on the first global data and then fuses the resulting data with the raw local projection data to obtain the second global data. In some embodiments, the first global data includes a first global image or a first global sinogram.

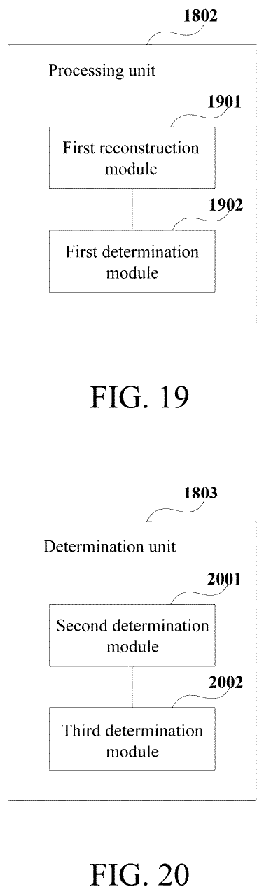

FIG. 19 is a schematic diagram of an implementation of a processing unit 1802 of the embodiments of the present application. As shown in FIG. 19, the processing unit 1802 includes a first reconstruction module 1901, configured to reconstruct the raw local projection data to obtain a first reconstructed image, and a first determination module 1902, configured to input the first reconstructed image into a pre-trained neural network model to obtain a first global image, and use the first global image as the first global data.

Implementations of the first reconstruction module 1901 and the first determination module 1902 may refer to 501-502, which will not be described herein again.

FIG. 20 is a schematic diagram of an implementation of a determination unit 1803 of the embodiments of the present application. As shown in FIG. 20, the determination unit 1803 includes a second determination module 2001, configured to perform a forward projection on the first global image to obtain a third global projection data or a third global sinogram, and a third determination module 2002, configured to fuse the raw local projection data and the third global sinogram to obtain the second global data, or fuse the raw local projection data and the third global projection data to obtain the second global data.

Implementations of the second determination module 2001 and the third determination module 2002 may refer to 601-602, which will not be described herein again.

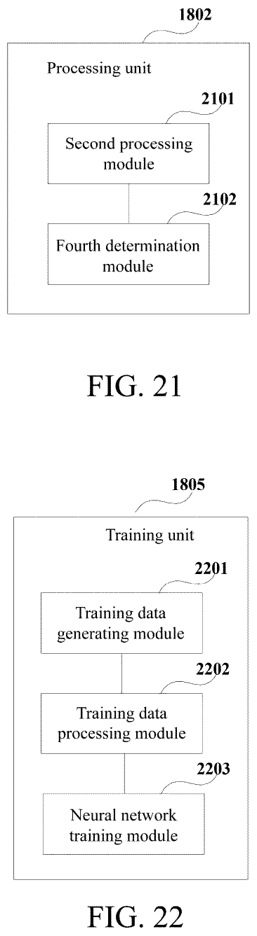

FIG. 21 is a schematic diagram of another configuration of the processing unit 1802 of the embodiments of the present invention. As shown in FIG. 21, the processing unit 1802 includes a second processing module 2101, configured to process the raw local projection data to obtain a first sinogram, and a fourth determination module 2102, configured to input the first sinogram into a pre-trained neural network model to obtain a first global sinogram, and use the first global sinogram as the first global data.

Implementations of the second processing module 2101 and the fourth determination module 2102 may refer to 901-902, which will not be described herein again.

In this embodiment, the determination unit 1803 fuses the first sinogram and the first global sinogram to obtain the second global data. In some embodiments, the apparatus further includes a training unit 1805.

FIG. 22 is a schematic diagram of a configuration of a training unit 1805 of the embodiments of the present application. As shown in FIG. 22, the training unit 1805 includes a training data generating module 2201, configured to acquire training global projection data and generate training local projection data according to the training global projection data, a training data processing module 2202, configured to process the training local projection data to obtain training input data, and process the training global projection data to obtain training output data, and a neural network training module 2203, configured to train the neural network model according to the training input data and the training output data.

Implementations of the training data generating module 2201, the training data processing module 2202, and the neural network training module 2203 may refer to 1201-1203, 1501-1505, and 1701-1704, which will not be described herein again.

In some embodiments, the training data processing module 2202 reconstructs the training local projection data to obtain a first training reconstructed image as the training input data, and reconstructs the training global projection data to obtain a second training reconstructed image as the training output, and the neural network training module 2203 trains the neural network model according to the first training reconstructed image and the second training reconstructed image. In some embodiments, the training data processing module 2202 fills first filling data in the training local projection data and then reconstructs the resulting data to obtain the first training reconstructed image; and the first filling data is determined according to projection data acquired by an edge detector module in the detector. In some embodiments, the first training reconstructed image and the second training reconstructed image are reconstructed images in a rectangular coordinate system after passing through a coordinate transformation.

In some embodiments, the training data processing module 2202 is further configured to take a first partial training image from the first training reconstructed image as the training input data, and take a second partial training image corresponding to the first partial training image from the second training reconstructed image as the training output data, wherein the size of the first partial training image is determined according to the position of the removed partial off-center detector modules, and the neural network training module 2203 trains the neural network model according to the first partial training image and the second partial training image. In some embodiments, the training data processing module 2202 is further configured to remove high-frequency information in the first partial training image and the second partial training image, and use the first partial training image that has had the high-frequency information removed as the training input data, and the second partial training image that has had the high-frequency information removed as the training output data, and the neural network training module 2203 trains the neural network model according to the first and second partial training images that have had the high-frequency information removed.

In some embodiments, the training data processing module 2202 processes the training local projection data to obtain a first training sinogram as the training input data, and the training global projection data to obtain a second training sinogram as the training output, and the neural network training module 2203 trains the neural network model according to the first training sinogram and the second training sinogram. In some embodiments, the training data processing module 2202 is further configured to divide the first training sinogram into a plurality of first training tiles of a predetermined size, and divide the second training sinogram into a plurality of second training tiles of a corresponding predetermined size, and use the first training tiles as the training input data, and the second training tiles as the training output data, and the neural network training module 2203 trains the neural network model according to the first training tiles and the second training tiles.

In some embodiments, the neural network training module 2203 trains the neural network model by using the training input data as an input to the neural network model, and the training output data as an output from the neural network model, or trains the neural network model by using the training input data as an input to the neural network model, and the difference between the training output data and the training input data as an output from the neural network model.

For simplicity, the above figures only exemplarily illustrate the connectional relationship or signal direction between various components or modules, but it should be clear to those skilled in the art that various related technologies such as bus connection can be used. The various components or modules can be implemented by means of a hardware facility such as a processor or a memory, etc. The embodiments of the present application are not limited thereto.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

It can be seen from the above embodiments that, the second global data is determined according to the raw local projection data and the first global data obtained by recovering the raw local projection data, and the second global data is reconstructed to obtain the diagnostic image, hence the detector data can be recovered, and the impact of artifacts due to data truncation can be reduced, and image quality can be guaranteed when the detector is incomplete.

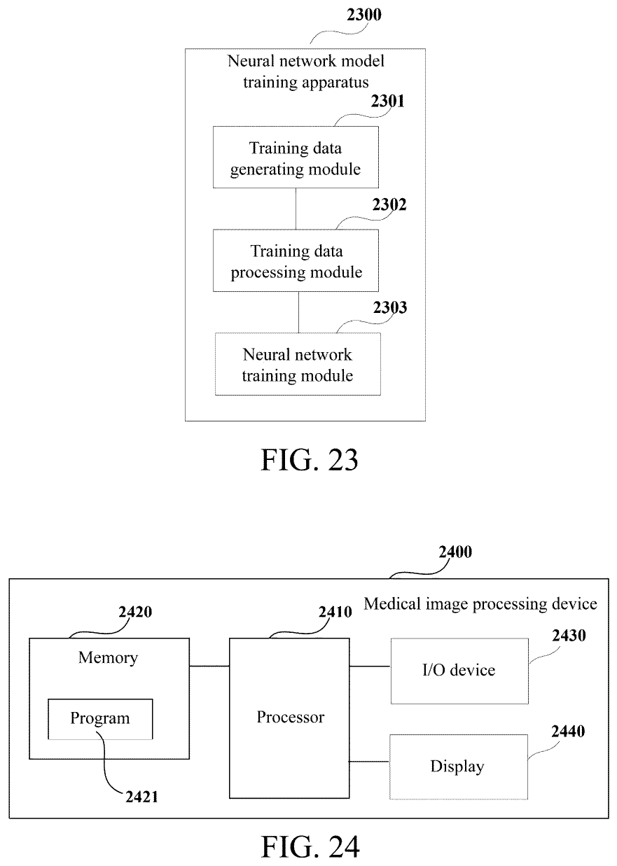

Further provided in the embodiments of the present application is an apparatus for training a neural network model, wherein the neural network model may be two-dimensional or three-dimensional. FIG. 23 is a schematic diagram of a neural network model training apparatus of the embodiments of the present application. As shown in FIG. 23, the apparatus 2300 includes a training data generating module 2301, configured to acquire training global projection data and generate training local projection data according to the training global projection data, a training data processing module 2302, configured to process the training local projection data to obtain training input related data, and process the training global projection data to obtain training output related data, and a neural network training module 2303, configured to train the neural network model according to the training input related data and the training output related data.

The implementation of the neural network model training apparatus 2300 may refer to the training unit 1805 in the aforementioned embodiments, which will not be described herein again one by one.

Further provided in the embodiments of the present application is a medical image processing device. FIG. 24 is a schematic diagram of a configuration of a medical image processing device of the embodiments of the present application. As shown in FIG. 24, the medical image processing device 2400 may include: one or more processors (for example, a central processing unit (CPU)) 2410, and one or more memories 2420 coupled to the one or more processors 2410. The memory 2420 can store image frames, neural network models, etc.; and in addition, it further stores a program 2421 for controlling an input device, and executes the program 2421 under control of the processor 2410. The memory 2420 may include, for example, a ROM, a floppy disk, a hard disk, an optical disk, a magneto-optical disk, a CD-ROM, or a non-volatile memory card.

In some embodiments, the functions of the medical image processing apparatus 1800 are integrated into the processor 2410 for implementation. The processor 2410 is configured to implement the medical image processing method as described in the aforementioned embodiments. For the implementation of the processor 2410, reference may be made to the aforementioned embodiments, which will not be described herein again. In some embodiments, the medical image processing apparatus 1800 and the processor 2410 are configured separately, for example, the medical image processing apparatus 1800 can be configured as a chip connected to the processor 2410 and the functions of the medical image processing apparatus 1800 can be achieved by means of the control of the processor 2410. In some embodiments, functions of the neural network model training apparatus 2300 are integrated into and implemented by the processor 2410. The processor 2410 is configured to implement the neural network model training method as described in the aforementioned embodiments. For the implementation of the processor 2410, reference may be made to the aforementioned embodiments, which will not be described herein again. In some embodiments, the neural network model training apparatus 2300 and the processor 2410 are configured separately, for example, the neural network model training apparatus 2300 can be configured as a chip connected to the processor 2410 and the functions of the neural network model training apparatus 2300 can be achieved by means of the control of the processor 2410.

In addition, as shown in FIG. 24, the medical image processing device 2400 may further include: an input device 2430 and a display 2440 (which displays a graphical user interface, and various data, image frames, or parameters generated in data acquisition and processing processes), etc., wherein the functions of the above components are similar to those in the prior art, which will not be described herein again. It should be noted that the medical image processing device 2400 does not necessarily include all of the components shown in FIG. 24. In addition, the medical image processing device 2400 may further include components not shown in FIG. 24, for which reference may be made to the related technologies.

The processor 2410 may be in communication with a medical device, the display, etc. in response to operation of the input device, and may also control input actions and/or state of the input device. The processor 2410 may also be referred to as a microcontroller unit (MCU), microprocessor or microcontroller or other processor apparatuses and/or logic apparatuses. The processor 2410 may include a reset circuit, a clock circuit, a chip, a microcontroller, and so on. The functions of the processor 2410 may be integrated on a main board of the medical device (e.g., the processor 2410 is configured as a chip connected to the main board processor (CPU)), or may be configured independently of the main board, and the embodiments of the present invention are not limited thereto.

Further provided in the embodiments of the present application is a medical device, the medical device including the medical image processing device 2400 of the aforementioned embodiments. The implementation of the medical image processing device 2400 is as described above, which will not be described herein again. In some embodiments, the medical device includes an electronic computed tomography device, but the present application is not limited thereto, and the medical device may also be other devices that may acquire medical imaging.

The functionality of the processor of the medical image processing device 2400 may be integrated into the main board of the medical device (e.g., the processor is configured as a chip connected to the main board processor (CPU)), or may be provided separately from the main board, and the embodiments of the present application are not limited thereto. In some embodiments, the medical device may further include other components. Please refer to the related technology for details, which will not be described herein again one by one.

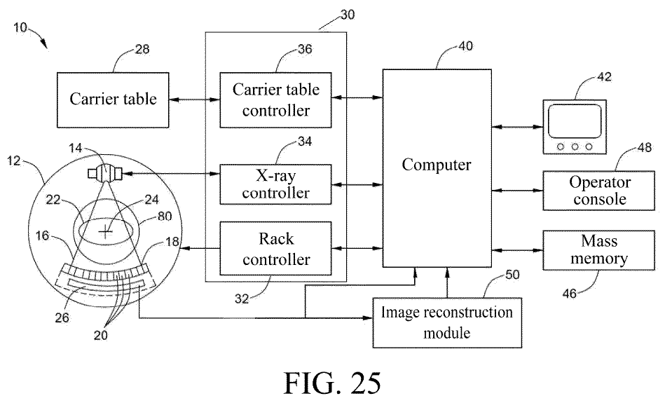

An example description is given below by taking the medical device being a CT device as an example. FIG. 25 is a schematic diagram of a CT system 10 of the embodiments of the present application. As shown in FIG. 25, the system 10 includes a rack 12. An X-ray source 14 and a detector 18 are disposed opposite to each other on the rack 12. The detector 18 is composed of a plurality of detector modules 20 and a data acquisition system (DAS) 26. The DAS 26 is configured to convert sampled analog data of analog attenuation data received by the plurality of detector modules 20 into digital signals for subsequent processing. The detector 18 is an incomplete detector.

In some embodiments, the system 10 is used for acquiring, from different angles, projection data of an object to be examined. Thus, components on the rack 12 are used for rotating around a rotation center 24 to acquire projection data. During rotation, the X-ray radiation source 14 is configured to emit X-rays 16 that penetrate the object to be examined toward the detector 18. Attenuated X-ray beam data is preprocessed and then used as projection data of a target volume of the object. An image of the object to be examined may be reconstructed on the basis of the projection data. The reconstructed image may display internal features of the object to be examined. These features include, for example, the lesion, size, and shape of body tissue structure. The rotation center 24 of the rack also defines the center of a scanning field 80.

The system 10 further includes an image reconstruction module 50. As described above, the DAS 26 samples and digitizes the projection data acquired by the plurality of detector modules 20. Next, the image reconstruction module 50 performs high-speed image reconstruction on the basis of the aforementioned sampled and digitized projection data. In some embodiments, the image reconstruction module 50 stores the reconstructed image in a storage device or a mass memory 46. Or the image reconstruction module 50 transmits the reconstructed image to a computer 40 to generate information for diagnosing and evaluating patients.

Although the image reconstruction module 50 is illustrated as a separate entity in FIG. 25, in some embodiments, the image reconstruction module 50 may form part of the computer 40. Or the image reconstruction module 50 may not exist in the system 10, or the computer 40 may perform one or more functions of the image reconstruction module 50. Furthermore, the image reconstruction module 50 may be located at a local or remote location and may be connected to the system 10 by using a wired or wireless network. In some embodiments, computing resources having a centralized cloud network may be used for the image reconstruction module 50.

In some embodiments, the system 10 includes a control mechanism 30. The control mechanism 30 may include an X-ray controller 34 configured to provide power and timing signals to the X-ray radiation source 14. The control mechanism 30 may further include a rack controller 32 configured to control the rotational speed and/or position of the rack 12 on the basis of imaging requirements. The control mechanism 30 may further include a carrier table controller 36 configured to drive a carrier table 28 to move to a suitable position so as to position the object to be examined in the rack 12, so as to acquire the projection data of the target volume of the object to be examined. Furthermore, the carrier table 28 includes a driving apparatus, and the carrier table controller 36 may control the carrier table 28 by controlling the driving apparatus.

In some embodiments, the system 10 further includes the computer 40, wherein data sampled and digitized by the DAS 26 and/or an image reconstructed by the image reconstruction module 50 is transmitted to a computer or the computer 40 for processing. In some embodiments, the computer 40 stores the data and/or image in a storage device such as a mass memory 46. The mass memory 46 may include a hard disk drive, a floppy disk drive, a CD-read/write (CD-R/W) drive, a digital versatile disc (DVD) drive, a flash drive, and/or a solid-state storage apparatus. In some embodiments, the computer 40 transmits the reconstructed image and/or other information to a display 42, the display 42 being communicatively connected to the computer 40 and/or the image reconstruction module 50. In some embodiments, the computer 40 may be connected to a local or remote display, printer, workstation and/or similar device, for example, connected to such devices of medical institutions or hospitals, or connected to a remote device by means of one or a plurality of configured wires or a wireless network such as the Internet and/or a virtual private network.

Furthermore, the computer 40 may provide commands and parameters to the DAS 26 and the control mechanism 30 (including the rack controller 32, the X-ray controller 34, and the carrier table controller 36), etc. on the basis of user provision and/or system definition, so as to control system operation, for example, data acquisition and/or processing. In some embodiments, the computer 40 controls system operation on the basis of user input. For example, the computer 40 may receive user input such as commands, scanning protocols and/or scanning parameters, by means of an operator console 48 connected thereto. The operator console 48 may include a keyboard (not shown) and/or touch screen to allow a user to input/select commands, scanning protocols and/or scanning parameters.

In some embodiments, the system 10 may include or be connected to an image storage and transmission system (PACS) (not shown in the figure). In some embodiments, the PACS is further connected to a remote system for example a radiology information system, a hospital information system, and/or an internal or external network (not shown) to allow operators at different locations to provide commands and parameters and/or access image data.

The method or process described in the aforementioned embodiments may be stored as executable instructions in a non-volatile memory in a computing device of the system 10. For example, the computer 40 may include executable instructions in the non-volatile memory and may apply the medical image processing method or neural network model training method in the embodiments of the present application.

The computer 40 may be configured and/or arranged for use in different manners. For example, in some implementations, a single computer 40 may be used; and in other implementations, a plurality of computers 40 are configured to work together (for example, on the basis of distributed processing configuration) or separately, wherein each computer 40 is configured to handle specific aspects and/or functions, and/or process data for generating models used only for a specific system 10. In some implementations, the computer 40 may be local (for example, in the same place as one or more systems 10, for example, in the same facility and/or the same local network); in other implementations, the computer 40 may be remote and thus can only be accessed by means of a remote connection (for example, by means of the Internet or other available remote access technologies). In a specific implementation, the computer 40 may be configured in a manner similar to that of cloud technology, and may be accessed and/or used in a manner substantially similar to that of accessing and using other cloud-based systems.

Once data (for example, a pre-trained neural network model) is generated and/or configured, the data can be replicated and/or loaded into the medical system 10, which may be accomplished in a different manner. For example, models may be loaded by means of a directional connection or link between the system 10 and the computer 40. In this regard, communication between different elements may be accomplished by using an available wired and/or wireless connection and/or according to any suitable communication (and/or network) standard or protocol. Alternatively or additionally, the data may be indirectly loaded into the system 10. For example, the data may be stored in a suitable machine-readable medium (for example, a flash memory card), and then the medium is used to load the data into the system 10 (for example, by a user or an authorized personnel of the system on site); or the data may be downloaded to an electronic device (for example, a laptop) capable of local communication, and then the device is used on site (for example, by a user or an authorized personnel of the system) to upload the data to the system 10 by means of a direct connection (for example, a USB connector).

Further provided in the embodiments of the present application is a computer readable program, wherein upon execution of the program, the program causes a computer to perform the medical image processing method or neural network model training method described in the aforementioned embodiments in the apparatus or medical device.

Further provided in the embodiments of the present application is a storage medium that stores a computer readable program, wherein the computer readable program causes a computer to perform the medical image processing method or neural network model training method d described in the aforementioned embodiments in the apparatus or medical device.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

The present application is described above with reference to specific embodiments. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application.

Preferred embodiments of the present application are described above with reference to the accompanying drawings. Many features and advantages of the implementations are clear according to the detailed description, and therefore the appended claims are intended to cover all these features and advantages that fall within the true spirit and scope of these implementations. In addition, as many modifications and changes could be easily conceived of by those skilled in the art, the embodiments of the present application are not limited to the illustrated and described precise structures and operations, but can encompass all appropriate modifications, changes, and equivalents that fall within the scope of the implementations.

1. A medical image processing apparatus, characterized by comprising:
an acquisition unit configured to acquire raw local projection data obtained by a detector after an object to be examined is scanned;
a processing unit configured to recover the raw local projection data to estimate first global data;
a determination unit configured to determine second global data according to the raw local projection data and the first global data; and
a reconstruction unit configured to reconstruct the second global data to obtain a diagnostic image.

2. The medical image processing apparatus according to claim 1, characterized in that the processing unit recovers the raw local projection data to obtain estimated missing data, and determines the first global data according to the estimated missing data and the raw local projection data.

3. The medical image processing apparatus according to claim 2, characterized in that the processing unit processes the raw local projection data to obtain a first reconstructed image or a first sinogram, and inputs the first reconstructed image or the first sinogram into a pre-trained neural network model to estimate the first global data.

4. The medical image processing apparatus according to claim 1, characterized in that the determination unit fuses the raw local projection data with the first global data to obtain the second global data.

5. The medical image processing apparatus according to claim 4, characterized in that the determination unit performs a forward projection on the first global data and then fuses the resulting data with the raw local projection data to obtain the second global data.

6. The medical image processing apparatus according to claim 1, characterized in that the first global data comprises a first global image or a first global sinogram.

7. The medical image processing apparatus according to claim 3, characterized by further comprising:
a training unit configured to train the neural network model by using training data, the training unit comprising:
a training data generating module configured to acquire training global projection data and generate training local projection data according to the training global projection data;
a training data processing module configured to process the training local projection data to obtain training input data, and process the training global projection data to obtain training output data; and
a neural network training module configured to train the neural network model according to the training input data and the training output data.

8. The medical image processing apparatus according to claim 7, characterized in that the training data processing module reconstructs the training local projection data to obtain a first training reconstructed image as the training input data, and reconstructs the training global projection data to obtain a second training reconstructed image as the training output data; and
the neural network training module trains the neural network model according to the first training reconstructed image and the second training reconstructed image.

9. The medical image processing apparatus according to claim 7, characterized in that the training data processing module processes the training local projection data to obtain a first training sinogram as the training input data, and processes the training global projection data to obtain a second training sinogram as the training output data; and
the neural network training module trains the neural network model according to the first training sinogram and the second training sinogram.

10. The medical image processing apparatus according to claim 8, characterized in that the training data processing module fills first filling data in the training local projection data and then reconstructs the resulting data to obtain the first training reconstructed image; and the first filling data is determined according to projection data acquired by an edge detector module in the detector.

11. The medical image processing apparatus according to claim 8, wherein the first training reconstructed image and the second training reconstructed image are reconstructed images in a rectangular coordinate system after passing through a coordinate transformation.

12. The medical image processing apparatus according to claim 8, characterized in that the training data processing module is further configured to take a first partial training image from the first training reconstructed image as the training input data, and take a second partial training image corresponding to the first partial training image from the second training reconstructed image as the training output data; and
the neural network training module trains the neural network model according to the first partial training image and the second partial training image.

13. The medical image processing apparatus according to claim 12, characterized in that the training data processing module is further configured to remove high-frequency information in the first partial training image and the second partial training image, and use the first partial training image that has had the high-frequency information removed as the training input data and the second partial training image that has had the high-frequency information removed as the training output data; and
the neural network training module trains the neural network model according to the first and second partial training images that have had the high-frequency information removed.

14. The medical image processing apparatus according to claim 9, characterized in that the training data processing module is further configured to divide the first training sinogram into a plurality of first training tiles of a predetermined size, and divide the second training sinogram into a plurality of second training tiles of a corresponding predetermined size, and use the first training tiles as the training input data, and the second training tiles as the training output data; and
the neural network training module trains the neural network model according to the first training tiles and the second training tiles.

15. The medical image processing apparatus according to claim 7, wherein the neural network training module trains the neural network model by using the training input data as an input to the neural network model and the training output data as an output from the neural network model, or trains the neural network model by using the training input data as an input to the neural network model and the difference between the training output data and the training input data as an output from the neural network model.

16. The medical image processing apparatus according to claim 1, characterized in that the detector is an incomplete detector having partial off-center detector modules removed from a plurality of detector modules arranged in an array.

17. A medical image processing method, characterized by comprising:
acquiring raw local projection data obtained by a detector after an object to be examined is scanned;
recovering the raw local projection data to estimate first global data;
determining second global data according to the raw local projection data and the first global data; and
reconstructing the second global data to obtain a diagnostic image.

18. The method according to claim 17, wherein the step of recovering the raw local projection data to estimate first global data comprises:
recovering the raw local projection data to obtain estimated missing data, and determining the first global data according to the estimated missing data and the raw local projection data.

19. A medical device, characterized by comprising the medical image processing apparatus according to claim 1.

20. The medical device according to claim 19, the medical device further comprising a detector, characterized in that the detector is an incomplete detector having partial off-center detector modules removed from a plurality of detector modules arranged in an array.
